(19) United States
(12) Patent Application Publication
(10) Pub. No.: US 2010/0221275 A1
(43) Pub. Date: Sep. 2, 2010

(54) PROTECTION OF AN ANIMAL AGAINST PESTIVIRUS INFECTION

(75) Inventors: Joe Brownlie, Hertfordshire (GB); Margaret Collins, South Wales (GB); Carole Thomas, Hertfordshire (GB); Ian Thompson, Basel (GB)

Correspondence Address: Novartis Animal Health US Inc., 3200 Northline Avenue, Suite 300, Greensboro, NC, 27408, US

(73) Assignee: Novartis AG, Basel (CH)

(21) Appl. No.: 12/682,931

(22) PCT Filed: Oct. 28, 2008
(86) PCT No.: PCT/EP2008/064601

§ 371 Date: Apr. 14, 2010

(30) Foreign Application Priority Data
Oct. 31, 2007 (EP) 07119742.0

(51) Int. Cl. A61K 39/295; A61K 39/12; A61K 39/00; A61P 31/04; A61P 31/12; A61P 37/04

(52) U.S. Cl. 424/186.1; 424/204.1; 424/192.1; 424/202.1; 424/201.1

(57) ABSTRACT

This invention involves compositions and methods for protecting animals from pestivirus infection and for treating animals infected with pestivirus. Pharmaceutical compositions containing E2 and NS3 from a pestivirus are used to protect animals or treat animals.

### FIELD OF INVENTION

This invention relates to the use of recombinant NS3 and recombinant E2 as a subunit vaccine to protect against pestivirus in animals, and more specifically, against bovine viral diarrhea virus (BVDV) infection in cattle.

### BACKGROUND OF INVENTION

Previous studies for subunit antigen vaccines against bovine viral diarrhea virus (BVDV) have focused on the E2 glycoprotein. It is well known that E2 stimulates virus neutralising antibodies (Donis, et al.; Neutralizing monoclonal antibodies to bovine viral diarrhea virus bind to the 56K to 58K glycoprotein; J Gen Virol 69 (Pt 1), 77-86 (1988)). Vaccination with E2 as a single antigen has given partial protective immunity although its highly variable sequence limits the cross-strain immunity induced. DNA vaccination with a plasmid encoding E2 did not induce serum neutralising antibodies against a heterologous virus, although after challenge neutralising titres were higher than those in naïve cattle indicating a priming effect to the heterologous infection (Harpin, et al.; Vaccination of cattle with a DNA plasmid encoding the bovine viral diarrhea virus major glycoprotein E2, J Gen Virol 80 (Pt 12), 3137-44 (1999)). DNA vaccination with E2 led to low levels of neutralising antibody against homologous virus with no neutralising antibodies to a heterologous strain (Nobiron, et al.; DNA vaccination against bovine viral diarrhea virus induces humoral and cellular responses in cattle with evidence for protection against viral challenge, Virology 21, 2082-92 (2003)).

Compared to E2, the non-structural proteins have much more conserved sequences across BVDV virus strains (Deng, R. & Brock, K. V.; Molecular cloning and nucleotide sequence of a pestivirus genome, noncytopathic bovine viral diarrhea virus strain SD-1, Virology 191, 867-9 (1992)). Because of this conservation of sequences, one non-structural protein, NS3, is combined with E2 in a subunit vaccine against BVDV and protects animals from infection.

Previously work was undertaken in cattle to evaluate an individually administered E2 DNA vaccine (Nobiron I., Thompson I., Brownlie J., & Collins M. E. (2003) DNA vaccination against bovine viral diarrhea virus induces humoral and cellular responses in cattle with evidence for protection against viral challenge, Vaccine 21, pp 2082-92.) and a NS3 DNA vaccine (Young N. J., Thomas C. J., Thompson I., Collins M. E. and Brownlie J. (2005) Immune responses to non-structural protein 3 (NS3) of bovine viral diarrhea virus (BVDV) in NS3 DNA vaccinated and naturally infected cattle. Prev. Vet. Med. 72, pp 115-120.). Neither of these DNA vaccines were able to protect bovine from BVDV infections. It is unclear if the reason for the lack on protection was the use of a DNA vaccine or if the antigens did not create a strong enough immune response. Whatever the reason for the failure, this work demonstrates that E2 and NS3, as individual antigens, did not protect cattle.

The inventors have created a new vaccine that provides protection to bovids against BVDV. This vaccine uses a combination of NS3 and E2 as protective antigens. This result is surprising considering that previous work demonstrated that each antigen alone was ineffective as a vaccine.

### BRIEF DESCRIPTION OF THE INVENTION

It is an object of this invention to have a vaccine or immunogenic composition which protects animals, preferably mammals, and more preferably cattle, from pestivirus infection, preferably from bovine viral diarrhea virus, classical swine fever virus and border disease virus, and more preferably from bovine viral diarrhea virus. This invention can be used to generate an immune response in cattle, sheep, rams, goats, pigs, and other mammals that are infected with pestivirus. It is an object of this invention that the vaccine contain E2 and NS3 proteins from bovine viral diarrhea virus or other pestivirus. The E2 and NS3 proteins can be produced recombinantly or purified from cells infected with the pestivirus. The vaccine can also contain antigens from other pathogens. The vaccine can contain E2 from non-cytopathic or cytopathic bovine viral diarrhea virus. The vaccine can contain NS3 from non-cytopathic or cytopathic bovine viral diarrhea virus. The amino acid sequence of NS3 can be from any pestivirus or can be the sequence of SEQ ID NO: 2. The amino acid sequence of E2 can be from any pestivirus or can be the sequence of SEQ ID NO: 4. E2 and NS3 can be a fusion protein or separate proteins.

It is an object of this invention to treat or prevent a disease caused by a pestivirus in a animal susceptible to infection by a pestivirus by administering to the animal a pharmaceutically effective amount of a composition containing E2 and NS3 proteins from pestivirus. The pestivirus can be bovine viral diarrhea virus, classical swine fever virus or border disease virus or any other pestivirus that is suitable. It is preferable that the pestivirus be bovine viral diarrhea virus. It is an object of this invention that the composition administered to the animal contains E2 and NS3 proteins from bovine viral diarrhea virus or another pestivirus. The E2 and NS3 proteins can be produced recombinantly or purified from cells infected with the pestivirus. The composition can contain adjuvants, diluents, and/or antigens from other diseases. The animals that are to receive the composition can be any animal, preferably mammal that is infected with a pestivirus. More preferably, the mammal will be sheep, rams, heifers, lambs, bulls, calves, sows, boars, piglets, and goats. NS3 and E2 can be derived from cytopathic or non-cytopathic BVDV or a combination thereof. The amino acid sequence of NS3 can be from any pestivirus or can be the sequence of SEQ ID NO: 2. The amino acid sequence of E2 can be from any pestivirus or can be the sequence of SEQ ID NO: 4. E2 and NS3 can be a fusion protein or separate proteins.

It is another object of this invention to use E2 and NS3 in the manufacture of medicament for preventing or treating pestivirus infection in an animal, more preferably a mammal, more preferably sheep, rams, heifers, lambs, bulls, calves, sows, boars, piglets, and goats. The pestivirus can be bovine viral diarrhea virus, classical swine fever virus or border disease virus. The E2 and NS3 can be purified from cells infected with the pestivirus or be recombinantly produced proteins. The amino acid sequence of NS3 can be from any pestivirus or can be the sequence of SEQ ID NO: 2. The amino acid sequence of E2 can be from any pestivirus or can be the sequence of SEQ ID NO: 4. E2 and NS3 can be a fusion protein or separate proteins.

It is an object of this invention to have a vaccine containing at least two antigens from a pestivirus where the antigens are preferably NS3 and E2. It is another object of this invention that the amino acid sequence of NS3 comprise the sequence in SEQ ID NO: 2 and that the amino acid sequence of E2 comprise the sequence in SEQ ID NO: 4. It is another object of this invention that antigens from other pathogens be included in this vaccine. Examples of the other pathogens include, but are not limited to, bovine herpes virus type 1 (BHV1), parainfluenza virus type 3 (PI3), bovine respiratory syncytial virus (BRSV), bovine influenza virus, Leptospira canicola, Leptospira grippotyphosa, Leptospira borgpetersenii hardjo-prajitno, Leptospira borgpetersenii hardjo-bovis, Leptospira icterohaemmorrhagia, Leptospira interrogans pomona, Leptospira bratislava, Campylobacter fetus, Mannheimia haemolytica, Pasteurella multocida, Mycobacterium bovis, Mycoplasma dispar, Mycoplasma bovis, Clostridium chauvoei, Clostridium haemolyticum, Clostridium septicum, Clostridium novyi, Clostridium perfringens type C, Clostridium perfringens type D, Clostridium sordellii, Clostridium tetani, Haemophilus somnus, Moraxella bovis, Escherichia coli, Salmonella typhimurium, Bacillus anthracis, Listeria monocytogenes, Actinomyces pyogenes, Ehrlichia bovis, Mycoplasma hyponeumoniae, Haemophilus parasuis, Pasteurella multocida, Streptococcus suis, Actinobacillus pleuropneumoniae, Bordetella bronchiseptica, Salmonella choleraesuis, Erysipelothrix rhusiopathiae, Leptospria ssp., Brachyspira pilosicoli, Brachyspira hyodysenteriae, Clostridium perfringens type A, swine influenza virus (SIV), porcine reproductive and respiratory syndrome virus (PRRSV), and porcine circovirus (PCV).

It is another object of this invention to have a vaccine against a pestivirus containing at least two expression vectors where one expression vector contains DNA encoding NS3 and the second expression vector contains DNA encoding E2. NS3 and E2 can be from any pestivirus, preferably from bovine viral diarrhea virus, classical swine fever virus and border disease virus, and more preferably from bovine viral diarrhea virus. It is preferable that the sequence of NS3 polynucleotide be the sequence of SEQ ID NO: 1 and that the sequence of the E2 polynucleotide be the sequence of SEQ ID NO: 3. It is another object of this invention that an adjuvant and/or antigens from other pathogens be included in this vaccine. The antigens from other pathogens can be proteins or DNA encoding proteins.

It is another object of this invention to have a vaccine against a pestivirus containing at least one expression vector where one expression vector contains DNA encoding both NS3 and E2. NS3 and E2 can be from any pestivirus, preferably from bovine viral diarrhea virus, classical swine fever virus and border disease virus, and more preferably from bovine viral diarrhea virus. It is preferable that the sequence of NS3 polynucleotide be the sequence of SEQ ID NO: 1 and that the sequence of the E2 polynucleotide be the sequence of SEQ ID NO: 3. It is another object of this invention that an adjuvant and/or antigens from other pathogens be included in this vaccine. The antigens from other pathogens can be proteins or DNA encoding proteins. It is an object of this invention that the NS3 and E2 be a fusion protein or, alternatively, separate proteins.

It is an object of this invention to have a method for preventing or treating pestivirus infection in an animal by administering NS3 and E2 to the animal. The NS3 and E2 can be administered as proteins, a fusion protein, one expression vector containing DNA which encodes both NS3 and E2, or two expression vectors where one expression vector contains DNA which encodes NS3 and the other expression vector contains DNA which encodes E2. It is preferable that the amino acid sequence for NS3 is the sequence of SEQ ID NO: 2, and that the amino acid sequence for E2 is the sequence of SEQ ID NO: 4. It is preferable that the DNA sequence of NS3 is the sequence of SEQ ID NO: 1, and that the DNA sequence of E2 is the sequence of SEQ ID NO: 3. It is another object of the invention that an adjuvant be combined with the proteins or expression vectors. It is another object of this invention that the proteins or expression vectors be administered at the same time at different sites on the animal, at the same time at the same site on the animal, at different times at the same site on the animal or at different times at different sites on the animal. If the proteins or expression vectors are administered at different times, then it is an object of this invention that the time period between the two dosages be less than four weeks apart, preferably less than two weeks apart, and more preferably less than one week apart.

### DETAILED DESCRIPTION OF THE INVENTION

Bovine viral diarrhea virus (BVDV) is a small, positive-sense, single stranded RNA virus in the family Flaviviridae and the genus Pestivirus. There are two different types of BVDV, cytopathic and non-cytopathic, and two major genotypes of BVDV, type 1 and type 2.

The present invention involves a “vaccine” (also called “immunogenic composition”) against pestivirus, and more preferably against BVDV. A vaccine is any composition which after administered to an animal induces an immune response in the animal which helps prevent the disease, or reduces the amount of symptoms of the disease, or helps cure the animal of the disease for which the vaccine is against. Vaccines can be used against bacteria, viruses, fungi, parasites, cancer, autoimmune diseases, and other types of diseases. The immune response can be transient or long-acting. Antigens within the vaccine can be in the form of proteins/polypeptides, DNA encoding proteins/polypeptides, or other compounds.

The present invention uses E2 and NS3 from cytopathic BVDV type 1, non-cytopathic BVDV type 1, cytopathic BVDV type 2, and/or non-cytopathic BVDV type 2, or a combination thereof. The present invention also uses E2 and NS3 from other pestiviruses, such as classical swine fever virus (CSFV) and border disease virus (BDV) to protect animals susceptible to those disease from those viruses. For this invention, one can use recombinantly produced E2 and NS3 proteins, E2 and NS3 purified from cells infected with a pestivirus, or DNA encoding E2 and NS3 to protect or treat an animal susceptible to a pestivirus infection, such as CSFV, BVDV, and BDV. Sheep, rams, heifers, bulls, sows, boars, and goats are among the animals for which the present invention can be used to treat or prevent an infection from BVDV, CSFV, and/or BDV.

The DNA sequence for BVDV NS3 used in the present invention is found in SEQ ID NO: 1. The amino acid sequence for BVDV NS3 used in the present invention is found in SEQ ID NO: 2. The DNA sequence of BVDV E2 used in the present invention is found in SEQ ID NO: 3. The amino acid sequence of BVDV E2 used in the present invention is found in SEQ ID NO: 4. The polynucleotides of the invention are the nucleic acid sequences for BVDV NS3 and E2, but can include the nucleic acid sequences of NS3 and E2 from other pestiviruses. The polypeptides of the invention are the amino acid sequences for BVDV NS3 and E2, but can include the amino acid sequences of NS3 and E2 from other pestiviruses.

The articles “a” and “an” are used herein to refer to one or to more than one (i.e., to at least one) of the grammatical object of the article. By way of example, “an element” means one element or more than one element.

An animal can be any mammal, fish, reptile, amphibian, or bird. Preferably, the animal is a mammal. Non-limiting examples of mammals include dog, cat, hamster, gerbil, rabbit, ferret, horse, cow, sheep, goat, deer, pig, monkey, and ape. Non-limiting examples of birds include chicken, goose, quail, duck, turkey, and parakeet.

A polynucleotide of the invention may comprise all, or a portion of, a subject nucleic acid sequence; a nucleotide sequence at least 60%, 70%, 80%, 90%, 95%, 96%, 97%, 98% or 99% (and every single digit between 60 and 100) identical to a subject nucleic acid sequence; a nucleotide sequence that hybridizes under stringent conditions to a subject nucleic acid sequence; nucleotide sequences encoding polypeptides that are functionally equivalent to polypeptides of the invention; nucleotide sequences encoding polypeptides at least about 60%, 70%, 80%, 85%, 90%, 95%, 98%, 99% (and every single digit between 60 and 100) homologous or identical with a subject amino acid sequence; nucleotide sequences encoding polypeptides having an activity of a polypeptide of the invention and having at least about 60%, 70%, 80%, 85%, 90%, 95%, 98%, 99% (and every single digit between 60 and 100) or more homology or identity with a subject amino acid sequence; nucleotide sequences that differ by 1 to about 2, 3, 5, 7, 10, 15, 20, 30, 50, 75 or more nucleotide substitutions, additions or deletions, such as allelic variants, of a subject nucleic acid sequence; nucleic acids derived from and evolutionarily related to a subject nucleic acid sequence; and complements of, and nucleotide sequences resulting from the degeneracy of the genetic code, for all of the foregoing and other nucleic acids of the invention. Nucleic acids of the invention also include homologs, e.g., orthologs and paralogs, of a subject nucleic acid sequence and also variants of a subject nucleic acid sequence which have been codon optimized for expression in a particular organism (e.g., host cell).

The term “operably linked”, when describing the relationship between two nucleic acid regions, refers to a juxtaposition wherein the regions are in a relationship permitting them to function in their intended manner. For example, a control sequence “operably linked” to a coding sequence is ligated in such a way that expression of the coding sequence is achieved under conditions compatible with the control sequences, such as when the appropriate molecules (e.g., inducers and polymerases) are bound to the control or regulatory sequence(s).

The term “regulatory sequence” is a generic term used throughout the specification to refer to polynucleotide sequences, such as initiation signals, enhancers, regulators and promoters, that are necessary or desirable to affect the expression of coding and non-coding sequences to which they are operably linked. Exemplary regulatory sequences are described in Goeddel; Gene Expression Technology: Methods in Enzymology, Academic Press, San Diego, Calif. (1990), and include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase (e.g., Pho5), the promoters of the yeast α-mating factors, the polyhedron promoter of the baculovirus system and other sequences known to control the expression of genes of prokaryotic or eukaryotic cells or their viruses, and various combinations thereof. The nature and use of such control sequences may differ depending upon the host organism. In prokaryotes, such regulatory sequences generally include promoter, ribosomal binding site, and transcription termination sequences. The term “regulatory sequence” is intended to include, at a minimum, components whose presence may influence expression, and may also include additional components whose presence is advantageous, for example, leader sequences and fusion partner sequences. In certain embodiments, transcription of a polynucleotide sequence is under the control of a promoter sequence (or other regulatory sequence) which controls the expression of the polynucleotide in a cell-type in which expression is intended. It will also be understood that the polynucleotide can be under the control of regulatory sequences which are the same or different from those sequences which control expression of the naturally-occurring form of the polynucleotide.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

Polypeptides or proteins of the invention include polypeptides or proteins containing all or a portion of a subject amino acid sequence; a subject amino acid sequence with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% (and every single digit between 60 and 100) identical or homologous to a subject amino acid sequence; and functional fragments thereof. Polypeptides and proteins of the invention also include homologs, e.g., orthologs and paralogs, of a subject amino acid sequence.

It is also possible to modify the structure of the polypeptides or proteins of the invention for such purposes as enhancing therapeutic or prophylactic efficacy, or stability (e.g., ex vivo shelf life, resistance to proteolytic degradation in vivo, etc.). Such modified polypeptides or proteins, when designed to retain at least one activity of the naturally-occurring form of the protein, are considered “functional equivalents” of the polypeptides or proteins described in more detail herein. Such modified polypeptides or proteins may be produced, for instance, by amino acid substitution, deletion, or addition, which substitutions may consist in whole or part by conservative amino acid substitutions.

For instance, it is reasonable to expect that an isolated conservative amino acid substitution, such as replacement of a leucine with an isoleucine or valine, an aspartate with a glutamate, a threonine with a serine, will not have a major affect on the biological activity of the resulting molecule. Whether a change in the amino acid sequence of a polypeptide results in a functional homolog may be readily determined by assessing the ability of the variant polypeptide to produce a response similar to that of the wild-type protein. Polypeptides in which more than one replacement has taken place may readily be tested in the same manner.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schinner., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). Examples of amino acid groups defined in this manner include: (i) a positively-charged group containing Lys, Arg and His, (ii) a negatively-charged group containing Glu and Asp, (iii) an aromatic group containing Phe, Tyr and Trp, (iv) a nitrogen ring group containing His and Trp, (v) a large aliphatic nonpolar group containing Val, Leu and De, (vi) a slightly-polar group containing Met and Cys, (vii) a small-residue group containing Ser, Thr, Asp, Asn, Gly, Ala, Glu, GIn and Pro, (viii) an aliphatic group containing Val, Leu, De, Met and Cys, and (ix) a small, hydroxyl group containing Ser and Thr.

The terms “polypeptide fragment” or “fragment”, when used in reference to a reference polypeptide or protein, refers to a polypeptide in which amino acid residues are deleted as compared to the reference polypeptide or protein, but where the remaining amino acid sequence is usually identical to the corresponding positions in the reference polypeptide or protein. Such deletions may occur at the amino-terminus or carboxy-terminus of the reference polypeptide or protein, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. A fragment can retain one or more of the biological activities of the reference polypeptide or protein. In certain embodiments, a fragment may comprise a domain having the desired biological activity, and optionally additional amino acids on one or both sides of the domain, which additional amino acids may number from 5, 10, 15, 20, 30, 40, 50, or up to 100 or more residues. Further, fragments can include a sub-fragment of a specific region, which sub-fragment retains a function of the region from which it is derived. In another embodiment, a fragment may have immunogenic properties.

The term “sequence homology” refers to the proportion of base matches between two nucleic acid sequences or the proportion of amino acid matches between two amino acid sequences. When sequence homology is expressed as a percentage, e.g., 50%, the percentage denotes the proportion of matches over the length of sequence from a desired sequence that is compared to some other sequence. Gaps (in either of the two sequences) are permitted to maximize matching; gap lengths of 15 bases or less are usually used, 6 bases or less are used more frequently, with 2 bases or less used even more frequently. The term “sequence identity” means that sequences are identical (i.e., on a nucleotide-by-nucleotide basis for nucleic acids or amino acid-by-amino acid basis for polypeptides) over a window of comparison. The term “percentage of sequence identity” is calculated by comparing two optimally aligned sequences over the comparison window, determining the number of positions at which the identical amino acids or nucleotides occurs in both sequences to yield the number of matched positions, dividing the number of matched positions by the total number of positions in the comparison window, and multiplying the result by 100 to yield the percentage of sequence identity. Methods to calculate sequence identity are known to those of skill in the art and described in further detail below.

The immunogenic composition of this invention could contain a fusion protein of NS3 and E2. A fusion protein refers to a chimeric protein as that term is known in the art and may be constructed using methods known in the art. In many examples of fusion proteins, there are two different polypeptide sequences, and in certain cases, there may be more. The polynucleotide sequences encoding the fusion protein may be operably linked in frame so that the fusion protein may be translated correctly. A fusion protein may include polypeptide sequences from the same species or from different species. In various embodiments, the fusion protein may contain one or more amino acid sequences linked to a first protein or polypeptide. In the case where more than one amino acid sequence is fused to a first protein or polypeptide, the fusion sequences may be multiple copies of the same sequence, or alternatively, may be different amino acid sequences. The fusion protein may be fused to the N-terminus, the C-terminus, or the N- and C-terminus of the first protein or polypeptide.

The term “isolated polypeptide” refers to a polypeptide, in certain embodiments prepared from recombinant DNA or RNA, or of synthetic origin or natural origin, or some combination thereof, which (1) is not associated with proteins that it is normally found with in nature, (2) is separated from the cell in which it normally occurs, (3) is free of other proteins from the same cellular source, (4) is expressed by a cell from a different species, or (5) does not occur in nature. It is possible for an isolated polypeptide to exist but not qualify as a purified polypeptide.

The term “isolated nucleic acid” and “isolated polynucleotide” refers to a polynucleotide whether genomic DNA, cDNA, mRNA, tRNA, rRNA, iRNA, or a polynucleotide obtained from a cellular organelle (such as mitochondria and chloroplast), or whether from synthetic origin, which (1) is not associated with the cell in which the “isolated nucleic acid” is found in nature, or (2) is operably linked to a polynucleotide to which it is not linked in nature. It is possible for an isolated polynucleotide to exist but not qualify as a purified polynucleotide.

The term “purified” refers to an object species that is the predominant species present (i.e., on a molar basis it is more abundant than any other individual species in the composition). A “purified fraction” is a composition wherein the object species is at least about 50 percent (on a molar basis) of all species present. In making the determination of the purity or a species in solution or dispersion, the solvent or matrix in which the species is dissolved or dispersed is usually not included in such determination; instead, only the species (including the one of interest) dissolved or dispersed are taken into account. Generally, a purified composition will have one species that is more than about 80% of all species present in the composition, more than about 85%, 90%, 95%, 99% or more of all species present. The object species may be purified to essential homogeneity (contaminant species cannot be detected in the composition by conventional detection methods) wherein the composition is essentially a single species. A skilled artisan may purify a polypeptide of the invention using standard techniques for protein purification in light of the teachings herein. Purity of a polypeptide may be determined by a number of methods known to those of skill in the art, including for example, amino-terminal amino acid sequence analysis, gel electrophoresis, mass-spectrometry analysis and the methods described herein.

The terms “recombinant protein” or “recombinant polypeptide” refer to a polypeptide or protein which is produced by recombinant DNA techniques. An example of such techniques includes the case when DNA encoding the expressed protein is inserted into a suitable expression vector which is in turn used to transform a cell to produce the protein or polypeptide encoded by the DNA.

In another aspect of the invention, the polynucleotide of the invention is provided in an expression vector containing a nucleotide sequence encoding a polypeptide of the invention and operably linked to at least one regulatory sequence. It should be understood that the design of the expression vector may depend on such factors as the choice of the host cell to be transformed and/or the type of protein desired to be expressed. The vector's copy number, the ability to control that copy number and the expression of any other protein encoded by the vector, such as antibiotic markers, should be considered.

An expression vector containing the polynucleotide of the invention can then be used as a pharmaceutical agent to treat an animal infected with BVDV, CSFV, and/or BDV or as a vaccine (also a pharmaceutical agent) to prevent an animal from being infected with BVDV, CSFV, and/or BDV, or to reduce the symptoms and course of the disease if the animal does become infected. One manner of using an expression vector as a pharmaceutical agent is to administer a nucleic acid vaccine to the animal at risk of being infected or to the animal after being infected. Nucleic acid vaccine technology is well-described in the art. Some descriptions can be found in U.S. Pat. No. 6,562,376 (Hooper et al.); U.S. Pat. No. 5,589,466 (Felgner, et al.); U.S. Pat. No. 6,673,776 (Felgner, et al.); and U.S. Pat. No. 6,710,035 (Felgner, et al.). Nucleic acid vaccines can be injected into muscle or intradermally, can be electroporated into the animal (see WO 01/23537, King et al.; and WO 01/68889, Malone et al.), via lipid compositions (see U.S. Pat. No. 5,703,055, Felgner, et al.), or other mechanisms known in the art field.

Expression vectors can also be transfected into bacteria which can be administered to the target animal to induce an immune response to the protein encoded by the nucleotides of this invention contained on the expression vector. The expression vector can contain eukaryotic expression sequences such that the nucleotides of this invention are transcribed and translated in the host animal. Alternatively, the expression vector can be transcribed in the bacteria and then translated in the host animal. The bacteria used as a carrier of the expression vector should be attenuated but still invasive. One can use Shigella spp., Salmonella spp., Escherichia spp., and Aeromonas spp., just to name a few, that have been attenuated but still invasive. Examples of these methods can be found in U.S. Pat. No. 5,824,538 (Branstrom et al.); U.S. Pat. No. 5,877,159 (Powell, et al.); U.S. Pat. No. 6,150,170 (Powell, et al.); U.S. Pat. No. 6,500,419 (Hone, et al.); and U.S. Pat. No. 6,682,729 (Powell, et al.).

Alternatively, the polynucleotides of this invention can be placed in certain viruses which act a vector. Viral vectors can either express the proteins of this invention on the surface of the virus, or carry polynucleotides of this invention into an animal cell where the polynucleotide is transcribed and translated into a protein. The animal infected with the viral vectors can develop an immune response to the proteins encoded by the polynucleotides of this invention. Thereby one can alleviate or prevent an infection by BVDV, CSFV, and/or BDV in the animal which received the viral vectors. Examples of viral vectors can be found U.S. Pat. No. 5,283,191 (Morgan et al.); U.S. Pat. No. 5,554,525 (Sondermeijer et al.) and U.S. Pat. No. 5,712,118 (Murphy).

The polynucleotide of the invention may be used to cause expression and over-expression of a polypeptide of the invention in cells propagated in culture, e.g. to produce proteins or polypeptides, including fusion proteins or polypeptides.

This invention pertains to a cell transfected with a recombinant gene in order to express a polypeptide of the invention. The cell may be any prokaryotic or eukaryotic cell. For example, a polypeptide of the invention may be expressed in bacterial cells, such as E. coli, insect cells (baculovirus), yeast, plant, or mammalian cells. Other suitable cells are known to those skilled in the art. Additionally, the cell may be supplemented with tRNA molecules not typically found in the organism so as to optimize expression of the polypeptide. Alternatively, the nucleotide sequence may be altered to optimize expression in the cell, yet the protein produced would have high homology to the originally encoded protein. Other methods suitable for maximizing expression of the polypeptide will be known to those in the art.

The present invention further pertains to methods of producing the polypeptides of the invention. For example, a cell transfected with an expression vector encoding a polypeptide of the invention may be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated.

A cell culture includes cells, media and other byproducts. Suitable media for cell culture are well known in the art. The polypeptide may be isolated from cell culture medium, cells, or both using techniques known in the art for purifying proteins, including ion-exchange chromatography, gel filtration chromatography, ultrafiltration, electrophoresis, and immunoaffinity purification with antibodies specific for particular epitopes of a polypeptide of the invention.

Thus, a nucleotide sequence encoding all or a selected portion of polypeptide of the invention, may be used to produce a recombinant form of the protein via microbial or eukaryotic cellular processes. Ligating the sequence into a polynucleotide construct, such as an expression vector, and transforming or transfecting into an organism, either eukaryotic (yeast, avian, insect or mammalian) or prokaryotic (bacterial cells), are standard procedures. Similar procedures, or modifications thereof, may be employed to prepare recombinant polypeptides of the invention by microbial means or tissue-culture technology.

Suitable vectors for the expression of a polypeptide of the invention include plasmids of the types: pTrcHis-derived plasmids, pET-derived plasmids, pBR322-derived plasmids, pEMBL-derived plasmids, pEX-derived plasmids, pBTac-derived plasmids and pUC-derived plasmids for expression in prokaryotic cells, such as E. coli. The various methods employed in the preparation of the plasmids and transformation of host organisms are well known in the art. For other suitable expression systems for both prokaryotic and eukaryotic cells, as well as general recombinant procedures, see Molecular Cloning, A Laboratory Manual, 2nd Ed., ed. by Sambrook, Fritsch and Maniatis (Cold Spring Harbor Laboratory Press, 1989) Chapters 16 and 17.

Alternatively, the polynucleotides of the invention which encode E2 and NS3 can be optimized for expression in plants (e.g., corn). The plant may be transformed with plasmids containing the optimized polynucleotides. Then the plant is grown, and the proteins of the invention are expressed in the plant, or the plant-optimized version is expressed. The plant is later harvested, and the section of the plant containing the proteins of the invention is processed into feed for the animal. This animal feed will impart immunity against a pestivirus when eaten by the animal. Examples of prior art detailing these methods can be found in U.S. Pat. No. 5,914,123 (Arntzen, et al.); U.S. Pat. No. 6,034,298 (Lam, et al.); and U.S. Pat. No. 6,136,320 (Arntzen, et al.).

One or more pharmacologically acceptable carriers can be added to the immunogenic compositions or vaccine of this invention. Examples of pharmaceutically acceptable carriers include, but are not limited to, solvents, dispersion media, coatings, adjuvants, stabilizing agents, diluents, preservatives, antibacterial and antifungal agents, isotonic agents, adsorption delaying agents, and the like. Diluents can include, but are not limited to, water, saline, dextrose, ethanol, glycerol (polypropylene glycol, polyethylene glycol, and others) and the like. Isotonic agents can include, but are not limited to, sodium chloride, dextrose, mannitol, sorbitol, lactose, and the like. Stabilizers can include, but are not limited to, albumin and the like.

Adjuvants can include, but are not limited to, the RIBI adjuvant system (Ribi Inc.), alum, aluminum salts, such as aluminum hydroxide and aluminum phosphate, aluminum hydroxide gel, cholesterol, oil-in-water emulsions, water-in-oil emulsions, water-in-oil-water (WOW) emulsions, block co-polymers, other polymers such as POLYGEN®, DEAE dextran, dextran sulfate, and methylacrylates, dimethylodecylammonium bromide, poxvirus proteins, Avridine lipid-amine adjuvant, lipid A, monophosoryl lipid A, animal oils (such as squalane and squalene), mineral oils (such as Drakeol and Montanides), vegetable oils (such as peanut, cottonseed, rapeseed, coconut oil), triterpenoid glycosides (such as saponin, Quil A, and QS21), detergents (such as Tween-80 and Pluronic), bacterial component adjuvants (such as Freund's incomplete adjuvant, Freund's complete adjuvant, Corynebacterium, Propionibacterium, Mycobacterium, heat labile enterotoxin from E. coli, and cholera toxin), interleukens, monokines, interferons, liposomes, ISCOMs, synthetic glycopeptides (such as muryamyl dipeptides and derivatives thereof), or combinations thereof.

The amount of E2 can range from about 1 μg to about 1 gram of protein per dose. The amount of NS3 can range from about 1 μg to about 1 gram of protein per dose. The amount of adjuvant can range from about 10 μg/ml to about 0.5 grams/ml. It is contemplated that antibiotics and/or antifungal agents are present in a range from about 1 μg/ml to about 0.5 grams/ml.

The compositions described in the present invention can be made in various forms depending on the route of administration. For example, the compositions can be made in the form of sterile aqueous solutions or dispersions suitable for injectable use, or made in lyophilized forms using freeze-drying techniques. Lyophilized compositions are typically maintained at about 4° C. and can be reconstituted in a stabilizing solution (such as saline and/or HEPES) with or without adjuvant, shortly prior to administration.

It is preferable that the compositions of the present invention be administered via an injection in intramuscular, subcutaneous, or subdermal routes. However, it is also possible that the compositions can be administered in other routes, such as oral, intranasal, intralymph node, intradermal, intraperitoneal, mucosal tissue uptake, rectal, transdermal, or vaginal routes of administration or a combination thereof.

The animal to be immunized can receive the compositions of this invention in a single dose or two or more doses which can be administered two to ten weeks apart. The compositions of this invention can be administered as a single dose or in a primer+booster fashion. The animal can be immunized prior to pregnancy, during pregnancy, or after pregnancy. The fetus and newborn of an animal vaccinated during pregnancy or prior to pregnancy may be protected for a certain amount of time after birth and then need to be vaccinated.

The vaccines or immunogenic compositions of this invention can optionally include antigens from other viruses, bacteria, parasites. Examples of such organisms include but are not limited to bovine herpes virus type 1 (BHV1), parainfluenza virus type 3 (PI3), bovine respiratory syncytial virus (BRSV), bovine influenza virus, Leptospira canicola, Leptospira grippotyphosa, Leptospira borgpetersenii hardjo-prajitno, Leptospira borgpetersenii hardjo-bovis, Leptospira icterohaemmorrhagia, Leptospira interrogans pomona, Leptospira bratislava, Campylobacter fetus, Mannheimia haemolytica, Pasteurella multocida, Mycobacterium bovis, Mycoplasma dispar, Mycoplasma bovis, Clostridium chauvoei, Clostridium haemolyticum, Clostridium septicum, Clostridium novyi, Clostridium perfringens type C, Clostridium perfringens type D, Clostridium sordellii, Clostridium tetani, Haemophilus somnus, Moraxella bovis, Escherichia coli, Salmonella typhimurium, Bacillus anthracis, Listeria monocytogenes, Actinomyces pyogenes, Ehrlichia bovis, and other pathogens of ruminants. Examples of porcine pathogens for which antigens can be combined include, but are not limited to, Mycoplasma hyponeumoniae, Haemophilus parasuis, Pasteurella multocida, Streptococcus suis, Actinobacillus pleuropneumoniae, Bordetella bronchiseptica, Salmonella choleraesuis, Erysipelothrix rhusiopathiae, Leptospria ssp., Brachyspira pilosicoli, Brachyspira hyodysenteriae, Clostridium perfringens type A, swine influenza virus (SIV), porcine reproductive and respiratory syndrome virus (PRRSV), and porcine circovirus (PCV).

Cells and Viruses

BVDV-free fetal bovine lung (FBL) cells are cultured in 10% MEM (Modified Eagles Medium containing Earles salts, Glutamax I and 25 mM HEPES (Invitrogen, Carlsbad, Calif.) and 10% foetal bovine serum (PAA Laboratories LTD, Somerset UK), 1% glutamine (Invitrogen) and 1% antibiotic/antimycotic (Invitrogen)) at 37° C. The Ky1203nc type 1a strain of BVDV (Clarke M. C., Brownlie J., and Howard C. J.; Isolation of cytopathic and non-cytopathic bovine viral diarrhea virus from tissues of infected animals. In Pestivirus Infections of Ruminants, pp 3-10. Edited by J. W. Harkness. Luxemburg: CEC (1987)), 456497 type 1a strain of BVDV (field isolate provided by the Veterinary Laboratories Agency), and cytopathic NADL type 1a (Collett, et al.; Proteins encoded by bovine viral diarrhea virus: The genomic organization of a pestivirus, Virology 165, 200-8 (1988)) are propagated in FBL cells.

Serum-free adapted Spodoptera frugiperda 9 (Sf9) cells are cultured in Sf900II medium with L-Glutamine (Invitrogen) containing 1% antibiotic/antimycotic (Invitrogen). Cells are maintained as suspension cultures, incubating at 28° C., rotating at 160 rpm.

Virus Titrations

Ten-fold serial dilutions of the virus stock is prepared in 2% MEM (Modified Eagles Medium containing Earles salts, Glutamax I and 25 mM HEPES (Invitrogen) and 2% foetal bovine serum (PAA), 1% glutamine (Invitrogen) and 1% antibiotic/antimycotic (Invitrogen)). Each dilution is transferred, 50 μl/well, to a column of a 96-well microtitre plate (Falcon, St. Louis, Mo.). FBL cells are added using 50 μl/well of a suspension containing 3×10⁵ cells/ml, in 2% MEM. The plates are incubated at 37° C. for 5 days.

An immunoperoxidase assay is carried out as follows: Cells are washed with warm MEM, are fixed with ice-cold 80% acetone (BDH), and are incubated at −20° C. for 30 minutes. All further wash steps use PBS-T (phosphate buffered saline containing 0.05% Tween-20 (Sigma, St. Louis, Mo.)), and all antibodies are diluted in 5% NRS (PBS-T containing 5% normal rabbit serum (Invitrogen)). Primary antibody V182 (hyperimmune serum raised against BVDV strain Ky1203nc) is diluted 1:100 and is added to cells for 30 minutes at 37° C. After 4 washes with PBS-T horseradish peroxidase conjugated anti-bovine IgG (Sigma) diluted 1:2000 is added for a further 30 minutes at 37° C. After washing, cells are developed with 100 μl/well AEC substrate. One AEC tablet (Sigma) is added to 1 ml DMF (Sigma) and is vortexed. Then 50 ml cold 0.05 M sodium acetate buffer (pH 5.0) is added. The solution is vortexed then is filtered twice through 0.45 μm filters, twice through 0.2 μm filters and 25 μl 30% H₂O₂ is added. After 15-30 minutes the cells are observed for cytoplasmic staining to confirm infection.

The proportion of infected wells is scored, and the titre is determined according to the Spearman-Karber calculation.

Immunoperoxidase assays are carried out to determine titres of the non-cytopathic viruses according to the above protocol. In the case of NADL, virus infection is evident by the presence of cytopathic effect.

Generation of E2 Baculovirus Expression Vector and Recombinant E2 Production

First, RNA is extracted from Ky1203ncp infected cells using RNA Stat-60 (AMS Biotechnology). One ml of reagent is used to homogenize 10⁷ cells before extraction with 0.2 ml of chloroform. RNA is precipitated from the aqueous phase with 0.5 ml isopropanol, washed with 75% ethanol, dried and resuspended in 15 μl DEPC treated sterile distilled water. Then, RNA is reverse transcribed to cDNA. 5 μl RNA is mixed with 1.5 μl pdN6 random hexamers (Pharmacia) and denatured at 70° C. for 10 minutes, then put on ice for 1 minute and left at room temperature for 5 minutes. Subsequently 1 μl of 10 mM dNTPS, 2 μl of DTT (0.1 mM), 4 μl of 5× buffer (Promega) and 20 units RNAsin (Promega) are added to the mixture and water to a final volume of 20 μl. This is preheated to 42° C. for 2 minutes before adding 200 units of Superscript II RT (Gibco) and incubating at 42° C. for 50 minutes. The enzyme is then inactivated by heating at 70° C. for 15 minutes.

Using the cDNA generated, the E2 coding sequence of Ky1203 ncp is amplified by polymerase chain reaction (PCR). PCR primers are designed to amplify the E2 region and incorporate a 6× histidine tag into the 3′sequence as well as appropriate restriction enzyme sites to enable cloning into the baculovirus transfer vector. Initially PCR is undertaken to amplify the E2 region of Ky1203ncp (primers sense 5′-CCGGATCCGCACCTAGACTGCAAACC-3′ (SEQ ID NO: 5); antisense 5′-CGCTGCAGTCAGGCGAAGTAATCCCG-3′ (SEQ ID NO: 6)). The PCR primers are designed to amplify the exact sequence of E2 deprived from its transmembrane anchor (1023 bp). Two rounds of PCR are performed with these primers. The first is performed with Taq polymerase, the second with Pfu polymerase. To a 5 μl sample of cDNA, one adds 2.5 μl of each forward and reverse primer (10 pmol/μl), 1 μl dNTPs (10 mM each), 5 μl of 10×Taq buffer, 3 μl of MgCl₂ (at 25 mM) and water to a final volume of 50 μl. Taq polymerase (5 U) (Promega) is then added to the mixture. When Pfu polymerase is used, MgCl₂ is included in the 10× buffer provided by the manufacturer. Denaturation is performed at 94° C. for 1 minute, annealing for 1 minute and elongation at 72° C. for 30 cycles. Elongation time varied from 1 minute per kb for Taq polymerase to 2 minute per kb for Pfu polymerase. The PCR product is cloned into pGEM3Zf(−) and the complete sequence determined. Using this construct as template, a subsequent version of the sequence is amplified in order to add a 3′ sequence coding for a histidine tag. This template is subsequently used in a PCR to amplify the E2 coding region using primers containing suitable restriction site for cloning into pMelBac. The PCR primers are sense 5′-CCGGATCCGCACCTAGACTGCAAACC-3′ (SEQ ID NO: 7); antisense 5′-GTCGACTCAATGATGATGATGATGATGGGCGAAGTAATCCCGG-3′ (SEQ ID NO: 8). The sense primer contains a BamHI cloning site; the antisense primer comprises a HIS-tag and SalI restriction enzyme site for cloning into pMelBac. This product is cloned into pT7blue2-T for sequencing and then digested to clone into linearised pMelBac using BamHI and SalI digestions. For restriction enzyme digests approximately 1 μg DNA is mixed with 2 μl of the supplied 10× buffer (Promega) in a final volume of 20 μl and digested with 10 U of enzyme. Incubation is performed for 2 hours at 37° C. Ligation is carried out using 50 ng of linearised vector DNA which is mixed with the insert to give a 1:3 molar ratio in a final volume of 10 μl with 1 μl of ligation buffer (Promega) and 1 μl of T4 DNA ligase (Promega). The mixture is incubated at 4° C. overnight for cohesive ends. After ligation the mixture is incubated with 200 μl thawed competent E. coli XLI blue cells on ice for 20 minutes. The mixture is then subjected to heat-shock at 42° C. for 45 seconds and the cells returned to ice for 2 minutes. The cells are then incubated in 800 μl SOC broth for 30 minutes at 37° C. in an orbital shaker incubator at 200 rpm before being plated onto LB-Agar plates supplemented with 100 μg/ml ampicillin. Individual colonies are picked and placed into 5 ml LB broth containing ampicillin and are grown overnight in orbital shaker (200 rpm) to enable mini-preparation of plasmid DNA using the Qiagen spin minprep kit. Restriction enzyme digests and DNA sequence analysis confirmed the integrity of the constructs.

The transfer vector containing E2 coding sequence is co-transfected into insect cells with baculoviral DNA (Invitrogen). Bac-N-Blue DNA (0.3 μg) is mixed with plasmid DNA (1.3 μg) (Baulovirus transfer vector pMelBac with the cloned cDNA), Grace's medium (0.6 ml) and Insectin liposomes (7 μl). The mixture is vortexed and incubated for 15 minutes at room temperature, then are carefully dropped on an insect cell monolayer previously seeded on a 35 mm dish. Following a 4 hour incubation period, 1 ml of complete TNM-FH is added to each dish and incubated at 27° C. for 3 days. Plaque assays are performed with 1 ml of several transfection supernatant dilutions added drop-wise to 100 mm dishes seeded with 5×10⁶ cells/dish and incubated for 1 hour at 27° C. After removing all the medium from the dish 5 ml of TNM-FH medium mixed with an equal volume of 2.5% baculovirus agarose is pipetted into 5 ml of TNM-FH supplemented with Xgal (150 μg/ml) and immediately poured onto the side of each dish. The dishes were placed in the incubator (27° C.) for 5-6 days.

Plaques are picked with a 1 ml pipette and the agarose plugs transferred into the well of a 12 well plate previously seeded with Sf21 insect cells (5×10⁵ cells/well). After incubation of 5 days half the P1 stock is used for PCR analysis whilst the rest is kept for the preparation of a higher titre stock. Two 25 cm² flasks seeded with 2×10⁶ insect cells are infected with 20 μl P1 stock and incubated at 27° C. for 8 days to generate P2 stocks. This is used to inoculate a large flask of log phase insect cells in order to obtain the P3 stock. Aliquots of this are maintained at 4° C. and stored at −70° C. A plaque assay is undertaken subsequently to obtain virus titre to enable infection of cultures at known MOI to be undertaken. PCR analysis on the P1 stocks is undertaken. The supernatants from the P1 stocks of several plaques are mixed with ice cold 20% polyethylene glycol in 1 M NaCl₂ and incubated at room temperature for 30 minutes. After centrifugation at 12,000 rpm for 10 minutes, the pellet is resuspended in sterile water (100 μl) with proteinase K (10 μl of 10 mg/ml solution) and incubated at 50° C. for 1 hour. DNA is extracted with an equal volumes of phenol/chloroform, centrifuged at 12,000 rpm for 5 minutes at room temperature and the aqueous phase transferred to a fresh tube. The DNA is mixed with 3 M sodium acetate, glycogen (5 μl of 10 mg/ml solution) and precipitated with 100% ethanol, incubated at −20° C. for 20 minutes and centrifuged at 4° C. for 15 minutes. The pellet is washed in 70% ethanol, air-dried and resuspended in 10 μl sterile water; 5 μl is used in a PCR reaction with primers that bind either side of the insertion site of the transfer vector giving a product of 839 bp for wild type virus, thereby allowing detection of plaque which contained only recombinant virus or a mixture of wild type and recombinant. Recombinant virus is grown to high titre stocks for use in expression studies.

Serum free adapted Sf9 cells are infected with E2 expressing baculovirus at a multiplicity of infection of 10 and incubated at 27° C. for 96 hours. The cell pellet is harvested by centrifugation at 1000 rpm for 10 minutes at 4° C. The cell pellet is treated with PBS containing 0.1 M Tris (pH 7.4) containing 1% Triton X-100 (Sigma) and 0.3% TNBP (Sigma) for 2 hours at 4° C. After centrifugation to remove cellular debris the lysate is stored at −70 C. The concentration of E2 is estimated by densitometric analysis of western blots. Each vaccine dose contained 100 μg E2 protein.

Generation of NS3 Baculovirus Expression Vector (pBlueBacHISNS3)

First, RNA is extracted from Ky1203ncp infected cells using RNA Stat-60 (AMS Biotechnology). One ml of reagent is used to homogenize 10⁷ cells before extraction with 0.2 ml of chloroform. RNA is precipitated from the aqueous phase with 0.5 ml isopropanol, washed with 75% ethanol, dried and resuspended in 15 μl DEPC treated sterile distilled water. Then, RNA is reverse transcribed to cDNA. 5 μl RNA is mixed with 1.5 μl pdN6 random hexamers (Pharmacia) and denatured at 70° C. for 10 minutes, then put on ice for 1 minute and left at room temperature for 5 minutes. Subsequently 1 μl of 10 mM dNTPS, 2 μl of DTT (0.1 mM), 4 μl of 5× buffer (Promega) and 20 units RNAsin (Promega) are added to the mixture and water to a final volume of 20 μl. This is preheated to 42° C. for 2 minutes before adding 200 units of Superscript II RT (Gibco) and incubating at 42° C. for 50 minutes. The enzyme is then inactivated by heating at 70° C. for 15 minutes.

Using the cDNA generated, the NS3 coding sequence of Ky1203 ncp is amplified by polymerase chain reaction (PCR). PCR primers are designed to amplify the NS3 region and incorporate a 6× histidine tag into the 3′sequence as well as appropriate restriction enzyme sites to enable cloning into the baculovirus transfer vector. Initially PCR is undertaken to amplify the NS3 region of Ky1203ncp (forward primer 5′-AACCATGGGGCCTGCCGTGTGTAAG-3′ (SEQ ID NO: X) and reverse primer 5′-AAGCGGCCGCAAACCCAGTCACTTGCTTCAG (SEQ ID NO: Y))

### Generation of NS3 S₁₆₃→A Mutation Baculovirus Expression Vector and Recombinant NS3 Production

The NS3 coding sequence comprising the S₁₆₃→A mutation is generated by inverse PCR. The primers are sense 5′-GAAAGGGTGGGCGGGCCTGC-3′ (SEQ ID NO: 9) and antisense 5′-AGGTTTTTTAGATCAAAGAAAGCCG-3′ (SEQ ID NO: 10)). 100 pmol of each primer is treated with polynucleotide kinase (5 U), mixed with 2 μl 10× kinase buffer, 4 μl 5 mM ATP and 3.5 μl distilled sterile water and incubated at 37° C. for 30 minutes before heating to 94° C. for 5 minutes. Inverse PCR is performed as follows 1 fmol template DNA (pBlueBacHISNS3) is mixed with 2 μl each kinased primer (5 pmol/ul), 10× Pfu buffer, 1 μl 10 mM dNTPs 0.5 μl Pfu and 34.9 μl distilled sterile water. The conditions of the PCR are 94° C. for 3 minutes, followed by 24 cycles of 94° C. for 1 minute, 52° C. for 1 minute and 72° C. for 14 minutes, followed by a final cycle of 94° C. for 1 minute, 52° C. for 1 minute and 72° C. for 20 minutes. At mid-point in the PCR the reaction it is paused and a further 0.5 μl Pfu is added to the reaction. The PCR product is resolved on an 1% agarose gel, and subsequently cut out from the gel and purified using a Qiaquick gel extraction kit. The weight of the gel slice is measured and then 3 volumes of buffer QG are added. After heating at 50° C. for 10 minutes, 1 volume of isopropanol is added, and the mixture is loaded onto a spin column. The spin column is spun for 1 minute at 13,000 rpm, and the flow through is discarded. Then 0.75 ml of PE solution is added, and the column is spun again for 1 minute at 13,000 rpm. The flow through is again discarded, and the column is spun once more as before. The DNA is eluted by adding 30 μl sterile distilled water into the column, waiting for 1 minute and then centrifuging for 1 minute at 13,000 rpm. The eluate, i.e., PCR product is stored at −20° C. The product of the PCR is subsequently ligated by mixing 100 ng of PCR product with 1 μl T4 ligase (Promega), 1 μl 10× ligase buffer and 6 μl of sterile water. After incubating at room temperature for four hours, half of the ligation mix is transformed into competent E. coli XLI-blue cells. Transformation is accomplished by heat shock at 42° C. for 45 seconds, and the cells returned to ice for 2 minutes. The cells are then incubated in 800 μl SOC broth for 30 minutes at 37° C. in an orbital shaker incubator at 200 rpm before being plated onto LB-Agar plates supplemented with 100 μg/ml ampicillin. Individual colonies are picked and placed into 5 ml LB broth containing ampicillin and are grown overnight in orbital shaker (200 rpm) to enable mini-preparation of plasmid DNA using the Qiagen spin minprep kit.

Several colonies are subsequently picked and minipreped, and the PCR directed mutation is confirmed by sequencing. The mutated NS3 is then ligated into pBlueBacHis2B by digesting with BamHI and PstI. The mutated NS3 construct generated from inverse PCR (20 μl) is digested with PstI (2 μl), in buffer H (5 μl) and 23 μl water. This reaction is incubated for 2 hours at 37° C. before heat inactivation at 65° C. for 20 minutes. Then the product is gel purified using Qiaquick gel extraction kit, and the product (20 μl) is digested with BamHI (2 μl) in the presence of 5 μl Buffer E (Promega) and 23 μl distilled water. Again the digest is performed at 37° C. for 2 hours and then heat inactivated for 20 minutes at 65° C. This reaction produces a 1.3 kb BamHI/BamHI insert and a 700 bp BamHI/PstI insert which are gel purified using Qiagen gel extraction kits. The recipient plasmid (pBlueBacHIS2B) is also digested with BamHI and PstI.

The 700 bp BamHI/Pst I insert is cloned into pBlueBacHis2B using the following protocol. 50 ng vector is incubated with either 50 ng or 150 ng of insert, 2 μl 10× ligase buffer (Promega), 0.5 μl T4 DNA ligase, and water to 20 μl. Ligations are incubated at 16° C. overnight. After transformation, clones are minipreped, and the insert is confirmed to be present by enzyme digestion with restriction endonuclease NdeI. The plasmid is then digested with BamHI (10 μl DNA, 2 μl BamHI, 5 μl 10× buffer and 33 μl water; 2 hours 37° C.) before adding shrimp alkaline phosphatase (SAP) to the digest (2 μl SAP buffer, 2 μl SAP enzyme and 16 μl water) and incubating for a further 30 minutes at 37° C. Then 2 μl of 0.5M EDTA is added at the mixture and heat inactivated at 65° C. for 20 minutes. The construct is gel purified and then the BamHI insert is ligated into this vector (50 ng vector, with 50 or 100 ng of BamHI digested insert with 2 μl 10× ligation buffer and 0.5 μl T4 DNA ligase, plus water to a final volume of 20 μl; incubated at 4° C. overnight). The ligation mixture (5 μl) is transformed into Invitrogen One Shot Top 10 E. coli cells as described previously, and several colonies are picked from the agar plate to grow up for extraction of plasmid DNA. A diagnostic digest of the final plasmid with Sal I (2 μl minprep DNA, 0.5 μl Sal I, 2 μl Buffer D (Promega) and 15 μl water) reveals two clones with correct orientation of insert. Sequencing confirms the integrity of the junctions; this construct is used for baculovirus cotransfection.

(Please Provide Protocol for Infection of Insect Cells with pBlueBacHISNS3 S₁₆₃→A Mutation.)

Following infection of insect cells with the recombinant baculovirus, purification of antigen is achieved via the histidine tag. Ni-NTA agarose (Qiagen) is used to enable purification of each antigen. Insect cells are harvested by centrifugation, 5 minutes at 1000 rpm, and then the cells are lysed in lysis buffer (containing 1% Igepal CA-630 and 10 mM imidazole) using 4 ml lysis buffer per 1-2×10⁷ cells and incubated on ice for 10 minutes. The lysate is centrifuged (10,000×g; 10 minutes 4° C.), and 200 μl 50% Ni-NTA slurry (Qiagen) per 4 ml of cleared lysate is added and mixed gently by rocking at 4° C. for 2-4 hours. The mixture is subsequently loaded into a column, the outlet cap is removed, and the resin column is washed twice with 800 μl wash buffer. Protein is eluted in 4×100 μl fractions of elution buffer and stored at −20° C. and analysed by protein assay.

Estimation of the protein concentration is carried out using the Pierce Protein Assay kit. A standard curve is prepared by making a 1/10 dilution of supplied BSA (2 mg/ml) and then in a 96 well plate making seven more 1:2 dilution down the first 2 columns of the plate using PBS as diluent. Samples of purified antigens are diluted 1:10, 1:100 and 1:1000, and then 100 μl of each antigen dilution are added to a quad of wells. A quad is also prepared of the blank. Substrate is prepared by mixing 2.5 ml reagent A with 2.4 ml reagent B and 0.1 ml reagent C (all supplied in kit) and then adding 100 μl to each well. After 2 hours at 37° C. the plates are read on a microtitre plate reader at 550 nm. Calculation of quantity of protein per sample is made via computational analysis from the standard curve data.

To analyse protein expression known quantities of protein or culture supernatant (approximately 20 μl) were mixed (1:1 vol/vol) with 2× SDS loading buffer prior to incubation at 100° C. for 5 minutes. SDS-PAGE (7.5%, 12% or 15% resolving gels) analyses are carried out on the Biometra minigel tank at 25 mA until the leading edge of the loading dye reaches the end of the gel (1-2 hours). Protein gels are either stained with Coomassie Brilliant Blue stain to visualise the protein bands directly or transferred to PVDF membrane (Sigma) using the Biometra fastblot for 45 minutes at 200 mA via Western Blot for specific immunodetection of the recombinant protein bands.

Membranes are blocked in 5% milk (PBS-T containing 5% skimmed milk powder (Marvel)) at 4° C. for 18 hours. After one wash in PBS-T the membrane is incubated at 37° C. for 2 hours with anti-BVDV hyperimmune serum V182 at 1:250 diluted in PBS-T containing 5% milk. After 4×10 minute washes in PBS-T, the secondary antibody is horseradish peroxidase conjugated anti-bovine IgG (Sigma) at 1:1000, also diluted in PBS-T containing 5% milk. After 4 washes, the membrane is developed with DAB peroxidase substrate (Sigma).

NS3-E2 Vaccine Preparation

Each dose of the NS3-E2 vaccine contains 100 μg NS3 and 100 μg E2 protein. The proteins are produced separately, as described above, and then the two proteins are combined together to yield a vaccine containing 200 μg of total protein.

Sf9 Cell Lysate Vaccine Preparation

Sf9 cells are pelleted by centrifugation at 1000 rpm, 10 minutes, 4° C., and are lysed in 0.1 M Tris (pH 7.4) containing 1% Triton X-100 and 0.3% TNBP for 2 hours at 4° C. After centrifugation, the lysate is stored at −70° C. A volume of lysate obtained from the same cell density as that used to produce 100 μg E2 is used for each vaccine dose.

Vaccination and Challenge

Fifteen BVDV naïve Holstein Friesian cattle (mixed sex) are divided into three groups (A, B, and C). Each animal receives a vaccine subcutaneously in the neck on days −63, −42 and −21. Each vaccine consisting of a 4 ml dose containing the appropriate antigen(s) in PBS containing 2.5% Quil A (Superfos Biosector, Denmark).

Group A receives 100 μg E2 and 100 μg NS3. Group B receives 100 μg E2. Group C receives 100 μg of Sf9 cell lysate. On day 0 all animals are challenged, by the intranasal route, with 6×10⁶ TCID₅₀/dose BVDV strain 456497. All animals are bled at each vaccination and on days 0, 3, 5, 6, 7, 10, 14 and 21 following challenge.

Virus Isolation from Buffy Coat and Nasal Mucosa

Whole blood and nasal swab samples are taken on days 0, 3, 5, 6, 7, 10 and 14 for virus isolation.

To isolate the buffy coat, 5 ml whole blood collected on EDTA is mixed with 45 ml ammonium chloride lysis buffer, is incubated at room temperature for 10 minutes, and then is centrifuged at 1000 rpm, 10 minutes, 4° C. The cell pellet is washed in 10 ml PBS, then resuspended in 1 ml 2% MEM.

Nasal swab samples are collected into nasal transport medium, vortexed, and are left to stand at room temperature for 15 minutes. The supernatant is used for virus isolation to minimise toxicity from the debris. To isolate virus, FBL cells are seeded at 2.5×10⁴ cells/well of a 24 well plate (Falcon) in 10% MEM. After 2 washes, samples are diluted 1:5 in 2% MEM, 0.5 ml added per well, and are incubated, in duplicate, for 2 hours at 37° C. A further 0.5 ml 2% MEM is added before incubation at 37° C. for 5 days. To minimise toxicity, nasal swab samples are aspirated and are replaced with 1 ml 2% MEM before incubation. Plates are subsequently frozen at −70° C. for 1 hour to lyse the cells, before monolayers from duplicate wells are resuspended, diluted 1:5 in 2% MEM and are added in duplicate to FBL cells seeded at 2.5×10⁴ cells/ml on coverslips using 0.5 ml/well of a 24 well plate. After 2 hours at 37° C., a further 0.5 ml 2% MEM is added before incubation at 37° C. for 5 days. Virus infection is detected by an immunofluorescence assay.

Immunofluorescent Assay to Detect Virus Infection

Cells are washed with warm MEM, are fixed with ice-cold 80% acetone and are incubated at −20° C. for 30 minutes. All further wash steps use PBS-T (phosphate buffered saline containing 0.05% Tween-20 (Sigma)), and all antibodies are diluted in 5% NRS (PBS-T containing 5% normal rabbit serum (Invitrogen)). Primary antibody V182 (hyperimmune serum raised against BVDV strain Ky1203nc) is diluted 1:100 and is added to cells for 30 minutes at 37° C. After 4 washes Cy3 labelled anti-bovine IgG (Stratech Scientific, Cambridgshire, UK) diluted 1:400 is added for a further 30 minutes at 37° C. After 4 washes in PBS-T and a final wash in H₂O, coverslips are mounted in fluorescent mounting medium (Dako, Glostrup, Denmark), and the cells are examined for cytoplasmic staining to confirm infection using the Olympus BX61 microscope.

Clinical Evaluation

Rectal temperatures are measured throughout challenge, and animals are scored on dyspnoea, coughing, nasal discharge and demeanour. Scores range from 1 (normal) to 3 (high clinical severity). Total leucocyte counts are performed on days −2 and −1 prior to challenge and on days 0, 3, 5, 6, 7, 10 and 14 after challenge on an automated haematological analyser (Abbott Cell-Dyn 3500; Abbott, Maidenhead, Berks, UK).

Indirect NS3 Antibody ELISA

The NS3 used in this assay is captured from a lysate obtained from BVDV-infected FBL cells. FBL cells in 850 cm² roller flasks (Falcon) at 70% confluence are infected with BVDV strain NADL using 10 μl, at 1×10⁶ TCID₅₀/ml, in 10 ml 2% MEM for 2 hours. Flasks are topped up with 50 ml 2% MEM and left until 50% of the cells showed cytopathogenicity. Cells are harvested by scraping, are resuspended in 1% Igepal (Sigma), and are lysed for one hour with agitation. After centrifugation at 1000 rpm for 15 minutes, the supernatant is stored at −70° C. Uninfected cells are also prepared in the same way as a negative background control.

All volumes are 50 μl/well, all incubations are at 37° C. for 2 hours and all washes between incubations are repeated 4 times with PBS-T unless stated otherwise. All samples and antibodies are diluted in 5% NPS (PBS-T containing 5% normal porcine serum (Invitrogen)).

A flat-bottomed 96-well plate (Falcon) is coated with a monoclonal antibody raised against BVDV NS2-3, WB112 (Veterinary Laboratories Agency), at 1:100 in carbonate coating buffer (Sigma) at 4° C. for 18 hours. The ELISA plate is blocked with 100 μl/well 5% NPS for 30 minutes at 37° C. before positive and negative control antigen, diluted 1:50, are added to alternate columns (positive and negative control antigens are supernatant from NADL infected and uninfected cells). Serum samples to be tested are added in quadruplicate, diluted 1:50. Biotinylated monoclonal anti-bovine IgG clone BG18 (Sigma) is added 1:40,000 followed by a further incubation with Streptavidin-horseradish peroxidase conjugate (Amersham Biosciences, Buckinghamshire, England) diluted 1:500 in PBS-T for one hour at 37° C. The ELISA is developed with OPD substrate (Sigma) and optical density measured using an Optimax microplate reader (Molecular Diagnostics, Sunnyvale, Calif.) at 490 nm using SOFTmax®PRO version 2.6.1.

To determine the immunoglobulin isotype of NS3 specific antibodies, the secondary antibody used is either horseradish peroxidase-conjugated anti-bovine IgG1 diluted 1:1818, horseradish peroxidase-conjugated anti-bovine IgG2 diluted 1:2222, or horseradish peroxidase-conjugated anti-bovine IgM diluted 1:4000 (Bethyl Laboratories, Montgomery, Tex.). The dilutions used enable optical densities for each isotype to be compared.

Direct E2 Antibody ELISA

All volumes used are 50 μl/well, all incubations are at 37° for 2 hours and all washes between incubation times are repeated 4 times with PBS-T, unless stated otherwise. All antibodies and samples are diluted in 5% NPS (PBS-T containing 5% normal porcine serum (Invitrogen)). A flat-bottomed 96-well plate (Falcon) is coated with HIS-tag purified E2 at a dilution of 1:50 (234 ng/well), in carbonate coating buffer (Sigma) at 4° C. for 18 hours. Non-specific binding is prevented by blocking with 100 μl/well 5% NPS for 30 minutes at 37° C. After one wash, serum samples diluted 1:100 are added in duplicate. Horseradish peroxidase-conjugated anti-bovine IgG (Sigma) is added at 1:5000. OPD substrate (Sigma) is used for development.

Virus Neutralisation

Serum samples are inactivated at 56° C. for 30 minutes, and then are mixed with 2% MEM (Minimal Essential Medium containing Earles salts, Glutamax I and 25 mM HEPES (Invitrogen), 2% foetal bovine serum (PAA), 1% glutamine (Invitrogen) and 1% antibiotic/antimycotic (Invitrogen)) and 50 μl are added to a 96 well plate. The concentration of samples range from 1:10 to 1:640 and 100 TCID₅₀ BVDV strain Ky1203nc and NADL are added per well. After incubation at 37° C. for 2 hours, 50 μl of FBL cells at a density of 3×10⁵ cells/ml in 2% MEM are added to each well and are incubated at 37° C., 5% CO₂ for 5 days.

An immunoperoxidase assay is carried out to determine infection with non-cytopathic virus. Cells are washed with warm MEM, are fixed with ice-cold 80% acetone (BDH) and are incubated at −20° C. for 30 minutes. All further wash steps use PBS-T (phosphate buffered saline containing 0.05% Tween-20 (Sigma)), and all antibodies are diluted in 5% NRS (PBS-T containing 5% normal rabbit serum (Invitrogen)). Primary antibody V182 (hyperimmune serum raised against BVDV strain Ky1203nc) is diluted 1:100 and is added to cells for 30 minutes at 37° C. After 4 washes with PBS-T, horseradish peroxidase conjugated anti-bovine IgG (Sigma) diluted 1:2000 is added for a further 30 minutes at 37° C. After washing, cells are developed with 100 μl/well AEC substrate. Briefly, one AEC tablet (Sigma) is added to 1 ml DMF (Sigma) and vortexed, and then 50 ml cold 0.05M sodium acetate buffer (pH 5.0) is added. The solution is vortexed then filtered twice through 0.45 μm filters, twice through 0.2 μm filters and 25 μl 30% H₂O₂ is added. After 15-30 minutes the cells are observed for cytoplasmic staining to confirm infection.

The dilution at which 50% of virus is neutralised, indicated by lack of infection in 50% of the wells calculated according to the Spearman-Karber method, is recorded as the neutralising titre of the sample.

Lymphocyte Proliferation to Recombinant Proteins

Proliferation assays are carried out on PBMCs isolated on day 0 and 21 using HIS-tag purified NS3, HIS-tag purified E2 and HIS-tag purified uninfected Sf9 cell supernatant, in triplicate, to a final concentration of 100 ng/ml. Purification of antigen is achieved via the histidine tag. Ni-NTA agarose (Qiagen) is used to enable purification of each antigen. His-tag protein are purified in accordance with the manufacturer's instructions and analysed by protein assay, SDS-PAGE and immunoblot detection.

To purify lymphocytes, blood collected on EDTA is mixed 1:1 with PBS and used to overlay 12 ml Histopaque 1083 (Sigma). This mixture is centrifuged at 1200 g for 45 minutes at 22° C. with low acceleration and no brake. The cells at the interface are collected, are added to 10 ml cold PBS, and are centrifuged at 1400 rpm at 4° C. for 15 minutes. Any remaining red blood cells are lysed by addition of 1 ml tissue-culture H₂O (Invitrogen), followed immediately by 10 ml PBS. After a further 2 washes with 10 ml PBS, purified lymphocytes are resuspended in RPMI 1640 (Sigma) containing 20% foetal bovine serum (PAA), 1% antibiotic/antimycotic (Invitrogen), 1% glutamine (Invitrogen), 50 μM 2-Mercaptoethanol (Invitrogen), and 10 mM sodium pyruvate (Sigma), and are seeded at 1×10⁵ cells/well of a flat-bottomed 96-well plate (Falcon). Individual histidine-tag purified proteins are added to a final concentration of 100 ng/ml in triplicate wells. Positive control wells are stimulated with Concanavilin A (Sigma) at 5 mg/ml. Negative control wells are stimulated with medium only. After 4 days at 37° C. in 5% CO₂, cells are labelled with 1 μCi ³H thymidine (Amersham Biosciences) per well. After 18 hours, cells are harvested onto a glassfibre filter (Perkin Elmer, Wellesley, Mass.) and the amount of incorporated thymidine is measured in a liquid scintillation analyser (Wallac MicroBeta TriLux, Perkin Elmer). Radioactive counts are recorded as corrected counts per minute (ccpm) and the stimulation index (SI) is calculated as the ratio between antigen-stimulated and medium-stimulated cells.

Antibody Responses After Vaccination

In the NS3-E2 vaccinates (group A), 4 out of 5 calves seroconvert to NS3 by the time of challenge on day 0. Two weeks post-challenge all calves in this group have a high antibody titre, demonstrating an anamnestic response. In the control group (group C) seroconversion to NS3 is observed in all calves 21 days post-challenge. The titres in this group are significantly lower than in the NS3-E2 vaccinates on day 0 (P=0.001), day 7 (P=0.008), day 14 (P=0.005) and day 21 (P=0.039). In the E2 vaccinates (group B), seroconversion to NS3 post-challenge is not evident in any calves.

All calves in the NS3-E2 and E2 vaccinated group seroconvert to E2 by day 0. There are no statistically significant differences between the mean E2 specific antibody titres in the NS3-E2 group compared to the E2 group either before vaccination on day −63 (P=0.4), day −42 (P=0.876), day −21 (P=0.864) and day 0 (P=0.948), or after challenge on day 7 (P=0.894), day 14 (P=0.674) and day 21 (P=0.712). The animals in the control group (group C) have low antibody titres to E2 even at 21 days after exposure to BVDV.

The immunoglobulin isotype of NS3 specific antibodies is predominantly IgG1 in all calves post-vaccination, with a lower concentration of IgM and IgG2. By day 21 post-challenge the level of IgG2 has risen significantly compared to the level of IgG2 at 0 and 7 days post-challenge. The levels of IgG1 and IgM remain similar at 21 days post-challenge to the levels at 7 days post-challenge. The isotype of E2 specific antibodies are both IgG1 and IgG2 in all calves post-vaccination. The concentration of IgG1 is higher with an IgG1:IgG2 ratio ranging from 1.8:1 to 4.6:1. The concentration of IgM is similar to IgG2. By day 21 post-challenge, the levels of IgG2 increases slightly with an IgG1:IgG2 ratio ranging from 1.4:1 to 2.4:1.

Serum neutralising antibodies to homologous virus are evident in the NS3-E2 and E2 groups after vaccination. Immediately prior to challenge the titres range from 1:160 to >1:640 in both groups while the serum neutralising titres of calves in the control group are all below 1:10. Neutralising antibody titres to heterologous virus are seen in the NS3-E2 and E2 groups after vaccination. The titres range from 1:80 to 1:640 in both groups. There are no neutralisation ability of samples from the control calves.

Memory Responses After Vaccination

On day 0 proliferation responses to recombinant NS3 are evident in 4 out of 5 NS3-E2 vaccinated calves. A response is also seen in one E2 vaccinated calf and one control calf, which is probably cross-reaction to insect cell material. Proliferation to recombinant E2 is seen in 3 out of 5 NS3-E2 vaccinated calves and one E2 vaccinated calf Responses seen 21 days post-challenge are much lower in the vaccinated groups with only 3 out of 5 NS3-E2 vaccinates responding to NS3 and one calf responding to E2. In the E2 vaccinates, only low responses are seen to both NS3 and E2. In the control group 3 out of 5 responded to NS3 with one calf demonstrating a stimulation index of over 70. The same calf is the only responder to E2 in the control group. There are no statistically significant differences between the mean stimulation index of the NS3-E2 and E2 vaccinates with stimulation to NS3 on day 0 (P=0.066) or day 21 (P=0.333) or with stimulation to E2 on day 0 (P=0.271) or day 21 (P=0.693). There are no statistically significant differences between the mean stimulation index of the E2 vaccinates and the controls with stimulation to either NS3 on day 0 (P=0.959) or day 21 (P=0.092) or E2 on day 0 (P=0.369) or day 21 (P=0.972). There is a statistically significant difference between the mean stimulation index of the NS3-E2 vaccinates and controls with stimulation to E2 on day 0 (P=0.028) but not day 21 (P=0.753), or with NS3 on day 0 (P=0.056) or day 21 (P=0.365).

Protection from Challenge

The mean number of animals from which virus can be isolated from the nasal mucosa is significantly higher in the control calves on day 5 (P=0.016) and day 6 (P=0.016) compared to the NS3-E2 vaccinates. Virus excretion from the nasal mucosa is completely prevented in 3 out of 5 NS3-E2 vaccinated calves, and virus is only detected in a fourth on a single day (Day 7). In the control group, all calves have detectable virus in nasal mucosa for between 2 and 5 days, with virus isolatable until Day 7 for 4 out of the 5 animals. In the E2 group all animals excrete virus from the nasal mucosa on at least one day, although the mean number of animals is not statistically significantly different to the NS3-E2 group. On day 5 and 6 virus isolation from the E2 group is significantly lower than the controls (P=0.050 and P=0.050 respectively).

Virus is only isolated from the buffy coat of one NS3-E2 vaccinate on Day 7, one E2 vaccinate on Day 7, and 2 control calves on one day only (Day 6 and 7, respectively). Virus isolation from buffy coat is not statistically significant between any groups, on any day.

There are no increase in mean rectal temperature in either the NS3-E2 or the E2 vaccinated group on any day after challenge but in the control group, the mean temperature is significantly higher on day 8 than both NS3-E2 (P=0.001) and E2 vaccinates (P=0.005). On further analysis it is evident that no animals in the NS3-E2 group experienced pyrexia, compared to the E2 group where one calf has a temperature of 40.1° C. on day 8, and the control group where all calves have a raised rectal temperature with 4 out of 5 calves being over 40° C.

Both vaccinated groups suffer some leucopenia 5 days after challenge. In the NS3-E2 vaccinates the extent of leucopenia is significantly lower on day 3 (P=0.008), day 6 (P=0.002) and day 14 (P=0.005) in comparison to the control group. There is no statistically significant difference between leucopenia in the NS3-E2 vaccinates compared to the E2 group.

All groups experience an increase in mean nasal discharge score, however the onset of nasal discharge in the NS3-E2 vaccinated group is delayed, in comparison to the controls. The control group has the first elevated score on day 5 post-challenge, and mean scores remain elevated for the remainder of the observations. Scores are significantly higher on day 5 (P=0.016) and day 9 (P=0.003) when compared to the NS3-E2 vaccinated group, and on day 9 (P=0.025) when compared to the E2 vaccinated group. There are no statistically significant differences between the NS3-E2 and E2 vaccinated groups.

Significant differences are observed between the control and both vaccinated groups following heterologous virus infection. Three calves in the NS3-E2 group demonstrate complete protection, shedding no virus from the nasal mucosa throughout the study. A fourth calf shows much reduced virus shedding compared to the controls although, disappointingly, virus can be isolated from the final calf for at least 5 days. Interestingly this calf has the lowest E2 antibody titre on the day of challenge. It also has one of the lowest neutralising titres, although a calf with the same titre shows no virus shedding from the nasal mucosa. In the E2 vaccinated group virus can be isolated from every calf for between one and 3 days, although the rate of virus clearance is faster and the period of virus establishment is less when compared to the control group. In the controls, all calves are viremic for at least 2 days with 2 calves excreting virus from the nasal mucosa for at least 5 days. There clearly is an advantage of co-delivery of NS3 with E2 in terms of reduced virus establishment in the nasal mucosa and increased rate of virus clearance in comparison to vaccination with E2 alone.

The characteristic pyrexia seen in acute BVDV infection around day 7-10 post-infection is not evident in the mean rectal temperatures of either of the vaccinated groups. When individual readings are studied, no calf in the NS3-E2 group experiences a rise in rectal temperature demonstrating complete absence of a major clinical sign of BVDV infection. In the E2 group one calf suffers a temperature increase of 40.1° C. possibly indicating that NS3 gives added protection in terms of limiting pyrexia. A further parameter indicating enhanced protection is the reduced severity of nasal discharge in the NS3-E2 group, whereby onset is delayed in comparison to the controls. The number of days in which elevated scores are seen was also less when compared to the E2 and control groups.

To investigate antigen specific T-cell responses lymphoproliferation assays are carried out on PBMCs, taken prior to and 21 days post-challenge, that are restimulated with recombinant E2 and NS3 protein. In the NS3-E2 vaccinated group, responses to both NS3 and E2 are observed in most calves prior to challenge. In the E2 vaccinated group only one calf shows proliferation to E2, with some cross-reaction to NS3. In accordance with these observations, proliferation to E2, after E2 DNA vaccination, is minimal 9 days post-challenge (Nobiron et al., 2003). The results of the current study demonstrate the surprising and expected enhancing effect of NS3 vaccination in terms of increased T-cell responses to E2 when compared to those seen in the E2 only vaccinated group. The responses seen on day 21 are much reduced in comparison to day 0.

Previous studies have shown proliferative responses after exposure to non-cytopathic BVDV have not increased until up to 63 days post-infection in naïve calves (Collen, T. & Morrison, W. I.; CD4(+) T-cell responses to bovine viral diarrhea virus in cattle, Virus Res 67, 67-80 (2000)). Furthermore, acute infection with non-cytopathic BVDV has been shown to strongly suppress an established lymphocyte proliferation response to Mycobacterium bovis bacilli Calmette-Guerin (Charleston, et al.; Masking of two in vitro immunological assays for Mycobacterium bovis (BCG) in calves acutely infected with non-cytopathic bovine viral diarrhea virus, Vet Rec 149, 481-4 (2001)). Without being bind bound to a particular theory, it is hypothesized that direct infection of lymphocytes with BVDV may impair their response and perhaps the PBMCs need time to recover after infection before the stimulating antigen is seen.

While this invention has been described with a reference to specific embodiments, it will be obvious to those of ordinary skill in the art that variations in these methods and compositions may be used and that it is intended that the invention may be practiced otherwise than as specifically described herein. Accordingly this invention includes all modifications encompassed within the spirit and scope of the invention as defined by the claims.

1-37. (canceled)

38. A vaccine comprising recombinant NS3 protein and recombinant E2 protein from a pestivirus.

39. The vaccine of claim 38 wherein the NS3 protein includes SEQ ID NO: 2 and the E2 protein includes SEQ ID NO: 4.

40. The vaccine of claim 38 wherein said NS3 and E2 are a fusion protein.

41. The vaccine of claim 38 wherein said pestivirus includes one or more of bovine viral diarrhea virus, classical swine fever virus and border disease virus.

42. The vaccine of claim 38 further comprising an adjuvant.

43. The vaccine of claim 38 further comprising an antigen from an organism selected from the group consisting of bovine herpes virus type 1 (BHV1), parainfluenza virus type 3 (PI3), bovine respiratory syncytial virus (BRSV), porcine influenza virus, Leptospira canicola, Leptospira grippotyphosa, Leptospira borgpetersenii hardjo-prajitno, Leptospira borgpetersenii hardjo-bovis, Leptospira icterohaemmorrhagia, Leptospira interrogans pomona, Leptospira bratislava, Campylobacter fetus, Mannheimia haemolytica, Pasteurella multocida, Mycobacterium bovis, Mycobacterium dispar, Mycoplasma dispar, Mycoplasma bovis, Clostridium chauvoei, Clostridium haemolyticum, Clostridium septicum, Clostridium novyi, Clostridium perfringens type C, Clostridium perfringens type D, Clostridium sordellii, Clostridium tetani, Haemophilus somnus, Moraxella bovis, Escherichia coli, Salmonella typhimurium, Bacillus anthracis, Listeria monocytogenes, Actinomyces pyogenes, Ehrlichia bovis, Mycoplasma hyponeumoniae, Haemophilus parasuis, Pasteurella multocida, Streptococcus suis, Actinobacillus pleuropneumoniae, Bordetella bronchiseptica, Salmonella choleraesuis, Erysipelothrix rhusiopathiae, Leptospria ssp., Brachyspira pilosicoli, Brachyspira hyodysenteriac, Clostridium perfringens type A, swine influenza virus (SIV), porcine reproductive and respiratory syndrome virus (PRRSV), and porcine circovirus (PCV).

44. The vaccine of claim 38 wherein said NS3 and E2 are derived from bovine viral diarrhea virus that includes one or both of a non-cytopathic bovine viral diarrhea virus and cytopathic bovine viral diarrhea virus

45. The vaccine of claim 38 wherein said recombinant NS3 includes the amino acid sequence of SEQ ID NO: 2.

46. The vaccine of claim 38 wherein said recombinant E2 includes the amino acid sequence of SEQ ID NO: 4.

47. A method for preventing or treating a disease caused by a pestivirus in an animal susceptible to infection with a pestivirus, comprising administering to said animal a pharmaceutically effective amount of a composition including recombinant NS3 protein and recombinant E2 protein from a pestivirus.

48. The method of claim 47, wherein the recombinant NS3 protein includes SEQ ID NO: 2 and the recombinant E2 protein includes SEQ ID NO: 4.

49. The method of claim 47 wherein said NS3 and E2 are a fusion protein.

50. The method of claim 47 wherein said pestivirus includes one or more of bovine viral diarrhea virus, classical swine fever virus and border disease virus.

51. The method of claim 47 wherein said composition further comprises an adjuvant.

52. The method of claim 47 wherein said composition further comprises at least one antigen from one other organism, said one other organism is selected from the group consisting of bovine herpes virus type 1 (BHV1), parainfluenza virus type 3 (PI3), bovine respiratory syncytial virus (BRSV), bovine influenza virus, Leptospira canicola, Leptospira grippotyphosa, Leptospira borgpetersenii hardjo-prajitno, Leptospira borgpetersenii hardjo-bovis, Leptospira icterohaeminorrhagia, Leptospira interrogans pomona, Leptospira bratislava, Campylobacter fetus, Mannheimia haemolytica, Pasteurella multocida, Mycobacterium bovis, Mycoplasma dispar, Mycoplasma bovis, Clostridium chauvoei, Clostridium haemolyticum, Clostridium septicum, Clostridium novyi, Clostridium perfringens type C, Clostridium perfringens type D, Clostridium sordellii, Clostridium tetani, Haemophilus somnus, Moraxella bovis, Escherichia coli, Salmonella typhimurium, Bacillus anthracis, Listeria monocytogenes, Actinomyces pyogenes, Ehrlichia bovis, Mycoplasma hyponeumoniae, Haemophilus parasuis, Pasteurella multocida, Streptococcus suis, Actinobacillus pleuropneumoniae, Bordetella bronchiseptica, Salmonella choleraesuis, Erysipelothrix rhusiopathiae, Leptospria ssp., Brachyspira pilosicoli, Brachyspira hyodysenteriae, Clostridium perfringens type A, swine influenza virus (SIV), porcine reproductive and respiratory syndrome virus (PRRSV), and porcine circovirus (PCV).

53. The method of claim 47 wherein said recombinant NS3 and recombinant E2 are derived from bovine viral diarrhea virus including one or both of non-cytopathic bovine viral diarrhea virus and cytopathic bovine viral diarrhea virus.

54. The method of claim 47 wherein said recombinant NS3 includes the amino acid sequence of SEQ ID NO: 2.

55. The method of claim 47 wherein said recombinant E2 includes the amino acid sequence of SEQ ID NO: 4.

56. A vaccine comprising one or more expression vectors including the nucleic acid sequences of SEQ ID NO: 1 and SEQ ID NO: 3.

57. The vaccine of claim 56 further comprising an adjuvant.

58. The vaccine of claim 56 wherein one expression vector contains the nucleic acid sequences of SEQ ID NO: 1 and SEQ ID NO: 3.

59. The vaccine of claim 56 wherein a first expression vector contains the nucleic acid sequence of SEQ ID NO: 1 and a second expression vector contains the nucleic acid sequence of SEQ ID NO: 3.

60. A method for preventing or treating a disease caused by a pestivirus in an animal susceptible to infection with a pestivirus, comprising administering to said animal a pharmaceutically effective amount of a composition comprising one or more expression vectors including the nucleic acid sequences of SEQ ID NO: 1 and SEQ ID NO: 3.

61. The method of claim 60 wherein said composition further comprises an adjuvant.

62. The method of claim 60 wherein one expression vector contains the nucleic acid sequences of SEQ ID NO: 1 and SEQ ID NO: 3.

63. The method of claim 60 wherein a first expression vector contains the nucleic acid sequence of SEQ ID NO: 1 and a second expression vector contains the nucleic acid sequence of SEQ ID NO: 3.

64. The method of claim 63 wherein said first expression vector and said second expression vector are administered at the same time in one or two sites.

65. The method of claim 63 wherein said first expression vector and said second expression vector are administered within a short time period of each other.

66. The method of claim 65 wherein said short time period is selected from the group consisting of less than one day, between one and seven days, and between one week and four weeks.


SEQUENCE LISTING (4 sequences)

SEQ ID NO: 1 - DNA, 2049 bp, Pestivirus
gggcctgccgtgtgtaagaagatcacagaacacgaaaaatgccacgtcaacatactagataaattaactgcatttttcgggattatgccgagggggactacacccagagccccagtgaggttccctacgagtttgttaaaagtgaggaggggtctggagactggttgggcttatacacaccaaggtgggataagttcagtcgaccatgtaaccgctggcaaagatctattggtctgtgacagcatgggaagaactagagtggtctgccaaagcaacaacaagttaactgatgagacagagtatggtgtaaagactgactcaggatgcccagatggtgccagatgttatgtgctgaatccagaggcagtcaacatatcgggatctaaaggggcagtcgtccaccttcaaaagacaggcggggaattcacgtgtgtcactgcatcaggcacaccggctttctttgatctaaaaaacctgaaagggtggtcgggcctgcctatatttgaagcctccagcggaagggtggttggtagggttaaggtggggaagaatgaagagtctaaacccacaaagataatgagtggtatccagaccgtctcaaagaacacagcagatctgactgagatggttaagaagatcaccagtatgaacaggggagacttcaagcagataactttagccacaggggcaggaaaaaccacagaacttccaaaagcagtgatagaggagacaggaagacacaagcgagtgctggtccttataccattgagggcggcggcagagtcagtttaccagtatatgagattgaaacacccgagtatctctttcaacctaaggataggggacatgaaagagggggacatggcgaccgggataacctatgcatcatacgggtacttctgccagatgcctcaaccaaagcttagagcagctatggtggaatactcatacatattcttagatgaataccactgtgccactcccgaacaactggcaatcatcggaaaaatccacagattttcagagagtataagagtagttgccatgactgctaccccggcagggtcagtaactacaacaggacaaaaacacccaatagaggaattcatagcccccggggtgatgaaaggggaagacctaggcagccagttccttgatatagcggggttaaaaatccccgtggatgagatgaaaggcaacatgttggttttcgtacccacgaggaacatggcagttgaggtggcaaagaagctaaaagctaagggctataattctgggtactattacagtggagaggatccagcaaatctgagagttgtaacatcacagtccccatatgtaattgtggccacgaatgccattgaatcaggagtgacattaccagatttggacacggtcgtggacacggggttgaaatgtgaaaagagggtgagggtatcatcgaagatacccttcattgtaacaggccttaagaggatggccgttactgtgggtgaacaggctcaacgtaggggcagggtaggtagagtgaaacccgggagatattacaggagccaggaaacagcaaccgggtcaaaggactaccactatgacctcttgcaggcacaaagatacggcattgaagacggaatcaacgtaacaaaatcctttagggaaatgaattatgactggagtctatatgtggaagacagcctgctaataacccaattagaaatactaaataatttactcatatcagaagatttgccagctgctgtcaagaatataatggccaggactgatcacccagagccgatccaacttgcatacaacagttacgaggtccaggtcccagtcctgttcccaaaaataaggaatggagaagtcacagacacctacgaaaactactcatttttaaacgctagaaagctaggagaggacgtacccacatacatctatgccactgaagatgaggacctggcagttgacctcctagggctagactggcccgatcctgggaatcagcaggtcgtggagactggtaaagcactgaagcaagtgactgggttt

SEQ ID NO: 2 - protein, 683 aa, Pestivirus
GPAVCKKITEHEKCHVNILDKLTAFFGIMPRGTTPRAPVRFPTSLLKVRRGLETGWAYTHQGGISSVDHVTAGKDLLVCDSMGRTRVVCQSNNKLTDETEYGVKTDSGCPDGARCYVLNPEAVNISGSKGAVVHLQKTGGEFTCVTASGTPAFFDLKNLKGWSGLPIFEASSGRVVGRVKVGKNEESKPTKIMSGIQTVSKNTADLTEMVKKITSMNRGDFKQITLATGAGKTTELPKAVIEETGRHKRVLVLIPLRAAAESVYQYMRLKHPSISFNLRIGDMKEGDMATGITYASYGYFCQMPQPKLRAAMVEYSYIFLDEYHCATPEQLAIIGKIHRFSESIRVVAMTATPAGSVTTTGQKHPIEEFIAPGVMKGEDLGSQFLDIAGLKIPVDEMKGNMLVFVPTRNMAVEVAKKLKAKGYNSGYYYSGEDPANLRVVTSQSPYVIVATNAIESGVTLPDLDTVVDTGLKCEKRVRVSSKIPFIVTGLKRMAVTVGEQAQRRGRVGRVKPGRYYRSQETATGSKDYHYDLLQAQRYGIEDGINVTKSFREMNYDWSLYVEDSLLITQLEILNNLLISEDLPAAVKNIMARTDHPEPIQLAYNSYEVQVPVLFPKIRNGEVTDTYENYSFLNARKLGEDVPTYIYATEDEDLAVDLLGLDWPDPGNQQVVETGKALKQVTGF

SEQ ID NO: 3 - DNA, 1023 bp, Pestivirus
cacctagactgcaaacctgaattctcatacgccatcgccaagaatgatagaattggcccattaggagctgaaggccttaccactacttggaaggattactcgcatgaaatgaagctggaagacacaatggtcatagcttggtgcaaagatggtgagtttctattcttcccaaggtgcacaagagaaactagataccttgcagttttgcattcaagagccttgccgaccagcgttgtattcaataaactttttgaggggcaaaggcaaggggacacagtcgaaatggaagacaactttgaatttggactctgcccatgtgatgccaaacccgtaataagagggaagttcaatacaacactgctaaacggaccggccttccagatggtatgccctataggatggacagggactgtgagctgtatgttagctaacagggataccctagacacagcagtagtacgggtgtataggaggtccaaaccattcccttatagacaagattgcatcactacaaaagttctaggggaggatctctataactgtgatcttggaggaaactggacttgtgtgactggagaccagctacaatacacaggaggccctattgaatcttgcaagtggtgtggttataaattccaaaaaagtgaggggttaccacactaccccattggcaagtgtaggttgaagaatgagacaggctacagatttgtagataacactgcttgtaacagagaaggtgttgccatagtacaacaaggattagtaaagtgtaagataggagacacagtcgtacaggtcatagctcttgataccgaacttgggcctatgccttgcaagccatatgagattatatcaagtgaggggcctgtggaaaagacggcttgtaccttcaactacacaaagacattgaaaaataaatattttgagcccagagacagttacttccagcaatacatgctaaaaggagattatcagtactggtttgacctggaagtcactgaccatcaccgggattacttcgcc

SEQ ID NO: 4 - protein, 341 aa, Pestivirus
HLDCKPEFSYAIAKNDRIGPLGAEGLTTTWKDYSHEMKLEDTMVIAWCKDGEFLFFPRCTRETRYLAVLHSRALPTSVVFNKLFEGQRQGDTVEMEDNFEFGLCPCDAKPVIRGKFNTTLLNGPAFQMVCPIGWTGTVSCMLANRDTLDTAVVRVYRRSKPFPYRQDCITTKVLGEDLYNCDLGGNWTCVTGDQLQYTGGPIESCKWCGYKFQKSEGLPHYPIGKCRLKNETGYRFVDNTACNREGVAIVQQGLVKCKIGDTVVQVIALDTELGPMPCKPYEIISSEGPVEKTACTFNYTKTLKNKYFEPRDSYFQQYMLKGDYQYWFDLEVTDHHRDYFA